(19) United States
(12) Patent Application Publication
(10) Pub. No.: US 2008/0082067 A1
(43) Pub. Date: Apr. 3, 2008

(54) Cellulose fibers having superabsorbent particles adhered thereto

(75) Inventors: S. Ananda Weerawarna, Seattle, WA (US); Su Bing, Federal Way, WA (US)

Correspondence Address: WEYERHAEUSER COMPANY;INTELLECTUAL PROPERTY DEPT., CH 1J27, P.O. BOX 9777, FEDERAL WAY, WA, 98063, US

(73) Assignee: Weyerhaeuser Co., Federal Way, WA (US)

(21) Appl. No.: 11/537,962

(22) Filed: Oct. 2, 2006

(51) Int. Cl. A61F 13/15

(52) U.S. Cl. 604/368

(57) ABSTRACT

Cellulose fibers having superabsorbent particles adhered thereto.

### BACKGROUND OF THE INVENTION

Personal care absorbent products, such as infant diapers, adult incontinent pads, and feminine care products, typically contain an absorbent core that includes superabsorbent polymer particles distributed within a fibrous matrix. Superabsorbents are water-swellable, generally water-insoluble absorbent materials having a high absorbent capacity for body fluids. Superabsorbent polymers (SAPs) in common use are mostly derived from acrylic acid, which is itself derived from petroleum oil, a non-renewable raw material. Acrylic acid polymers and SAPs are generally recognized as not being biodegradable. Despite their wide use, some segments of the absorbent products market are concerned about the use of non-renewable petroleum oil derived materials and their non-biodegradable nature. Acrylic acid based polymers also comprise a meaningful portion of the cost structure of diapers and incontinent pads. Users of SAP are interested in lower cost SAPs. The high cost derives in pail from the cost structure for the manufacture of acrylic acid which, in turn, depends upon the fluctuating price of petroleum oil. Also, when diapers are discarded after use they normally contain considerably less than their maximum or theoretical content of body fluids. In other words, in terms of their fluid holding capacity, they are “over-designed”. This “over-design” constitutes an inefficiency in the use of SAP. The inefficiency results in part from the fact that SAPs are designed to have high gel strength (as demonstrated by high absorbency under load or AUL). The high gel strength (upon swelling) of currently used SAP particles helps them to retain a lot of void space between particles, which is helpful for rapid fluid uptake. However, this high “void volume” simultaneously results in there being a lot of interstitial (between particle) liquid in the product in the saturated state. When there is a lot of interstitial liquid the “rewet” value or “wet feeling” of an absorbent product is compromised.

In personal care absorbent products, U.S. southern pine fluff pulp is commonly used in conjunction with the SAP. This fluff is recognized worldwide as the preferred fiber for absorbent products. The preference is based on the fluff pulp's advantageous high fiber length (about 2.8 mm) and its relative ease of processing from a wetland pulp sheet to an airlaid web. Fluff pulp is also made from renewable and biodegradable cellulose pulp fibers, Compared to SAP, these fibers are inexpensive on a per mass basis, but tend to be more expensive on a per unit of liquid held basis. These fluff pulp fibers mostly absorb within the interstices between fibers. For this reason, a fibrous matrix readily releases acquired liquid on application of pressure. The tendency to release acquired liquid can result in significant skin wetness during use of an absorbent product that includes a core formed exclusively from cellulosic fibers. Such products also tend to leak acquired liquid because liquid is not effectively retained in such a fibrous absorbent core.

Superabsorbent composites in fiber form have a distinct advantage over particle forms in some applications. Such superabsorbent composite fibers can be made into a pad form directly. Liquid acquisition will be more uniform compared to a fiber pad with shifting superabsorbent particles.

A need therefore exists for fibrous superabsorbent materials that have the ability to have superabsorbent particles attached to the fibers. Biodegradable renewable fibers such as cellulose fiber is ideally suitable for such a fibrous superabsorbent composite material, if it can be treated and made to have strong affinity to superabsorbent particles. In this way, the superabsorbent material can be used in absorbent product designs that are efficient. These and other objectives are accomplished by the invention set forth below.

### SUMMARY OF THE INVENTION

The invention provides cellulose fibers having superabsorbent particles adhered thereto, comprising cellulose fibers treated with a hydrophilic polysaccharide polymer and having superabsorbent particles adhered thereto. The particles are selected from the group consisting of synthetic polymers, carboxyalkyl cellulose polymers, carboxyalkyl starch polymers, alginates, chitosans, and starches.

In one embodiment, the invention provides cellulose fibers having superabsorbent particles adhered thereto, comprising cellulose fibers treated with a hydrophilic polysaccharide polymer and a crosslinking agent, wherein the treated fibers have superabsorbent particles adhered thereto. The particles are selected from the group consisting of synthetic polymers, carboxyalkyl cellulose polymers, carboxyalkyl starch polymers, alginates, chitosans, and starches.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a scanning electron microscope photograph (13×) of representative fibers with adhered superabsorbent particles of the invention (Sample 3, Table 1);

FIG. 2 is a scanning electron microscope photograph (100×) of representative fibers with adhered superabsorbent particles of the invention (Sample 3, Table 1);

FIG. 3 is a scanning electron microscope photograph (13×) representative fibers with adhered superabsorbent particles of the invention (Sample 4, Table 1); and

FIG. 4 is a scanning electron microscope photograph (100×) of representative fibers with adhered superabsorbent particles of the invention (Sample 4, Table 1).

### DETAILED DESCRIPTION OF THE INVENTION

The present invention provides cellulose fibers having superabsorbent particles adhered thereto. Methods for making the cellulose fibers having superabsorbent particles adhered thereto are described.

The cellulose fibers having superabsorbent particles adhered thereto, include cellulose fibers treated with a hydrophilic polysaccharide polymer.

Although available from other sources, suitable cellulosic fibers are derived primarily from wood pulp. Suitable wood pulp fibers for use with the invention can be obtained from well-known chemical processes such as the kraft and sulfite processes, with or without subsequent bleaching. Pulp fibers can also be processed by thermomechanical, chemithermomechanical methods, or combinations thereof. A high alpha cellulose pulp is also a suitable wood pulp fiber. The preferred pulp fiber is produced by chemical methods. Ground wood fibers, recycled or secondary wood pulp fibers, and bleached and unbleached wood pulp fibers can be used. Softwoods and hardwoods can be used. Suitable fibers are commercially available from a number of companies, including Weyerhaeuser Company. For example, suitable cellulosic fibers produced from southern pine that are usable with the present invention are available from Weyerhaeuser Company under the designations CF416, NF405, PL416, FR516, and NB416. Other suitable fibers include northern softwood and eucalyptus fibers.

As used herein, the term “hydrophilic polysaccharide polymer” refers to any one of a variety of polysaccharide polymers that are hydrophilic and that have a tendency to associate with cellulose. Representative hydrophilic polysaccharide polymers include natural polymers, such as galactomannan polymers, glucomannan polymers, alginic acids, carageenans, starches and starch derivatives such as carboxymethyl starch, and hydroxyethyl starch, and cellulose derivatives such as such as carboxymethyl cellulose, hydroxyethyl cellulose. In one embodiment, the hydrophilic polysaccharide polymer is a galactomannan polymer. Representative galactomannan polymers include guar gum, locust bean gum, and tara gum. In one embodiment, the hydrophilic polysaccharide polymer is a glucomannan polymer. Representative glucomannan polymers include konjac gum. In one embodiment, the polysaccharide is guar gum. The cellulose treated with hydrophilic polysaccharide polymer includes from about 1 to about 20 percent by weight hydrophilic polysaccharide polymer based on the weight of cellulose.

The preparation of representative cellulose fibers treated with a hydrophilic polysaccharide polymer (e.g., guar gum treated cellulose fibers) is described in Example 1. In general, hydrophilic polysaccharide polymer treated cellulose is prepared by dissolving a desired amount of the hydrophilic polysaccharide polymer in water (e.g., 10 g in 1000 mL water) to provide a solution and then adding cellulose fibers (e.g., 100 g) with mixing to provide a suspension. The treated fibers are obtained by filtration and drying (e.g., 10% by weight hydrophilic polysaccharide polymer treated cellulose).

The polysaccharide treated fiber in the product cellulose fibers are present in an amount form about 20 to 50% by weight of the product fibers,

In one embodiment, the cellulose fibers having superabsorbent particles adhered thereto, include cellulose fibers treated with a hydrophilic polysaccharide polymer and a crosslinking agent (added prior to the addition of superabsorbent particles). Suitable crosslinking agents include crosslinking agents that are reactive toward hydroxyl groups and carboxyl groups. Representative crosslinking agents include metallic crosslinking agents, such as aluminum (III) compounds, titanium (IV) compounds, bismuth (III) compounds, boron (III) compounds, and zirconium (IV) compounds. The numerals in parentheses in the preceding list of metallic crosslinking agents refers to the valency of the metal. Metallic crosslinking agents provide metal ion crosslinks.

Representative metallic crosslinking agents include aluminum sulfate; aluminum hydroxide; dihydroxy aluminum acetate (stabilized with boric acid); other aluminum salts of carboxylic acids and inorganic acids; other aluminum complexes, such as Ultrion 8186 from Nalco Company (aluminum chloride hydroxide); boric acid; sodium metaborate; ammonium zirconium carbonate (AZC); zirconium compounds containing inorganic ions or organic ions or neutral ligands; bismuth ammonium citrate (BAC); other bismuth salts of carboxylic acids and inorganic acids; titanium (IV) compounds, such as titanium (IV) bis(triethylaminato) bis(isopropoxide) (commercially available from the Dupont Company under the designation Tyzor TE); and other titanates with alkoxide or carboxylate ligands.

The crosslinking agent is applied in an amount up to about 20 percent by weight based on the total weight of the treated cellulose fibers. The amount of first crosslinking agent applied to the treated cellulose will vary depending on the crosslinking agent. In general, the fibers have an aluminum content up to about 2.0% by weight based on the weight of the composite fibers for aluminum crosslinked fibers, a titanium content of up to about 4.5% by weight based on the weight of the composite fibers for titanium crosslinked fibers, a zirconium content of up to about 6.0% by weight based on the weight of the composite fibers for zirconium crosslinked fibers; and a bismuth content up to about 5.0% by weight based on the weight of the composite fibers for bismuth crosslinked fibers.

The cellulose fibers of the invention include adhered superabsorbent particles. Suitable particles include those derived from synthetic hydrophilic polymers (e.g., superabsorbent polymers or SAPs), such as polyacrylic acids, polyacrylamides, and polyaspartic acids; and hydrophilic polymers (e.g., superabsorbent polymers) derived natural polymers, such as celluloses (e.g., carboxymethyl cellulose), alginates, chitosans, and starches (e.g., carboxymethyl starch). The combination of a carboxyalkyl cellulose and either a glucomannan or galactomannan polymer is not considered to be a superabsorbent particle in the context of this invention.

Superabsorbent particles in the product cellulose fibers are be present in an amount form about 50 to about 80% by weight of the product fibers.

The product fibers are highly absorptive. The fibers have a Free Swell Capacity of from about 30 to about 60 g/g (0.9% saline solution), a Centrifuge Retention Capacity (CRC) of from about 15 to about 35 g/g (0.9% saline solution), and an Absorbency Under Load (AUL) of from about 15 to about 30 g/g (0.9% saline solution).

The product fibers are useful as a superabsorbent in personal care absorbent products (e.g., infant diapers, feminine care products and adult incontinence products). The fibers have the ability to absorb water, saline solutions and biological fluids such as urine and the fibrous form also helps in wicking. The fibers are useful in a variety of other applications, including, for example, wound dressings, cable wrap, absorbent sheets or bags, and packaging materials.

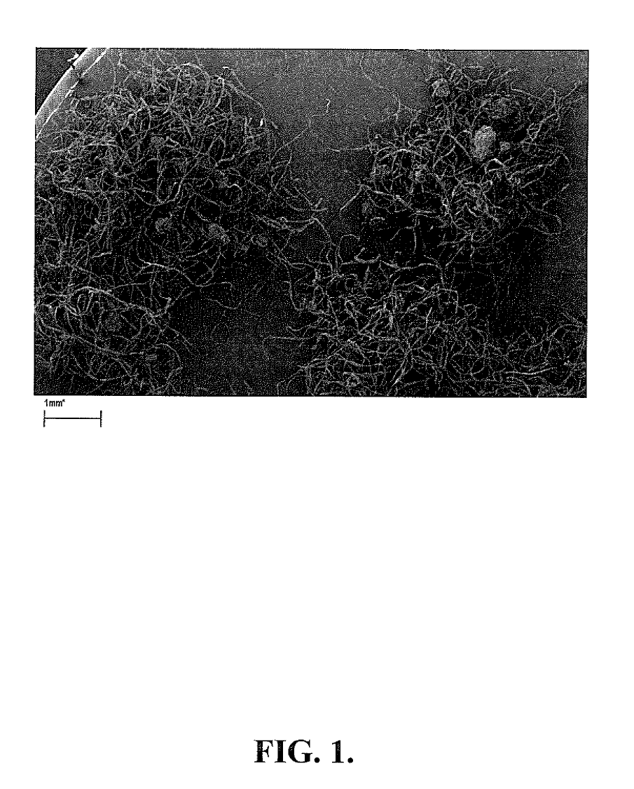

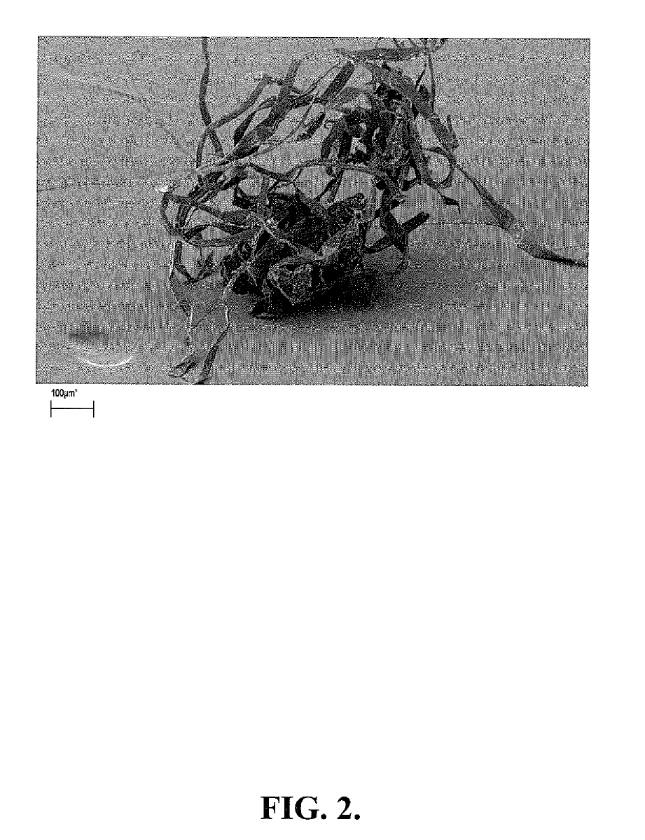

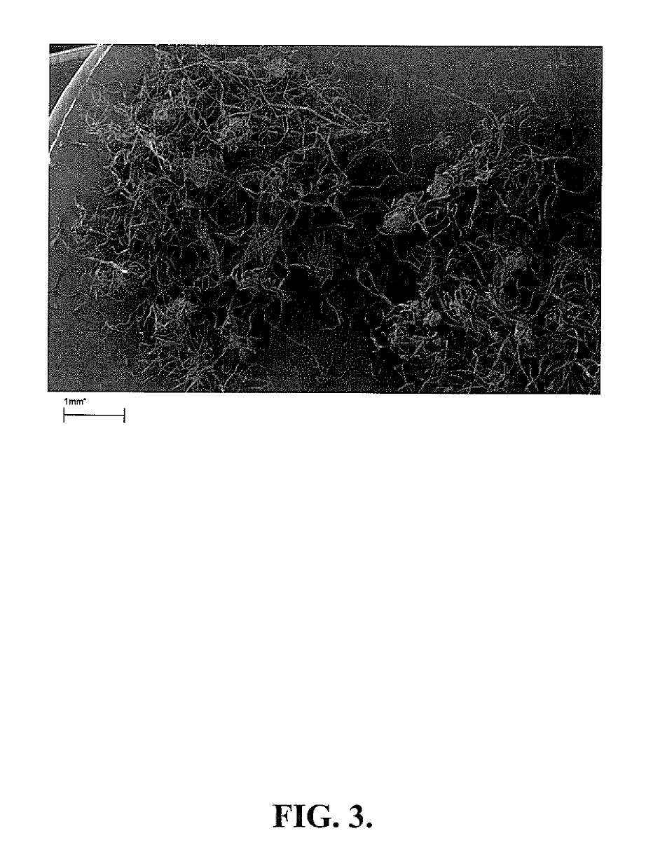

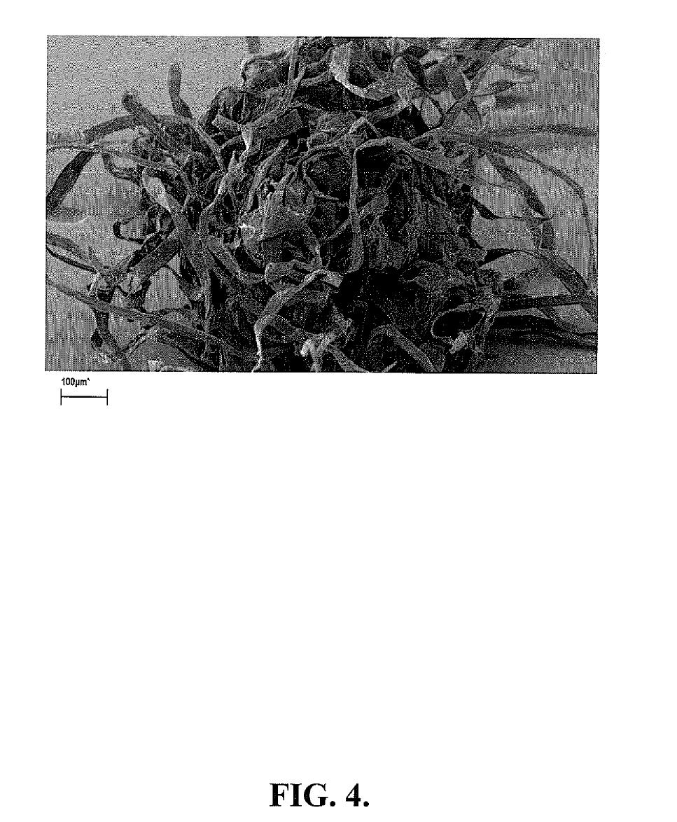

Representative cellulose fibers having superabsorbent particles adhered thereto are shown in FIGS. 1-4. FIG. 1 is a scanning electron microscope photograph (13×) of representative cellulose fibers having adhered superabsorbent particles (Sample 3, Table 1). FIG. 2 is a scanning electron microscope photograph (100×) of representative cellulose fibers having adhered superabsorbent particles (Sample 3, Table 1). FIG. 3 is a scanning electron microscope photograph (13×) of representative cellulose fibers having adhered superabsorbent particles (Sample 4, Table 1). FIG. 4 is a scanning electron microscope photograph (100×) of representative cellulose fibers having adhered superabsorbent particles (Sample 4, Table 1).

The preparations of representative fibers are described in Examples 2 and 3. The composition and liquid absorbent characteristics of representative fibers are summarized in the Table 1. In Table 1, for the superabsorbent particle and the polysaccharide polymer treated cellulose, the values in parentheses refer to the relative weight of each in the composite superabsorbent fiber (wgt % total wgt); “Crosslinking agent/4 g” refers to the amount of crosslinking agent applied per 4 g product; “SANIWET-4500” refers to a synthetic superabsorbent particle (a polyacrylic acid particle) commercially available from Hoechst Celanese; “NKS pulp with 10% GG” refers to northern kraft spruce (NKS) pulp treated with 10 weight % guar gum; and “with wash” refers to washing the treated fibers with 100% ethanol or 100% isopropanol before drying.

The cellulose fibers having adhered superabsorbent particles of the invention can be made by a method that includes the steps of adding a plurality of particles to a first aqueous mixture comprising cellulose treated with a polysaccharide polymer to provide a second aqueous mixture; and mixing the second aqueous mixture with a water-miscible solvent to provide cellulose fibers having particles adhered thereto. In one embodiment, the method includes applying a crosslinking agent to the cellulose treated with the polysaccharide in the first aqueous mixture prior to adding the particles.

The product fibers are obtained by filtration. In one embodiment, the method further includes the steps of drying the cellulose fibers having adhered particles to provide partially-dried composite fibers (30-50% consistency). The partially-dried composite fibers can be fiberized to provide partially-dried fiberized composite fibers. The partially-dried fiberized composite fibers can be further dried to provide dried fiberized cellulose fibers having particles adhered thereto.

As noted above, the fibers can be prepared by a process that includes optionally treating an aqueous mixture of a plurality of superabsorbent particles and cellulose treated with a hydrophilic polysaccharide polymer with a metal crosslinking agent to provide a mixture, and then further mixing the mixture with a water-miscible solvent.

When a crosslinking agent is optionally used before adding superabsorbent particles to the aqueous solution containing the cellulose fiber treated with the hydrophilic polysaccharide polymer, the agent provides additional crosslinking of the polymers of the superabsorbent particles. This is suitable when the particles are not sufficiently crosslinked (or under crosslinked by design). When the particles are highly crosslinked, this additional crosslinking is not used to prevent loss of absorbent capacity of the product composite fibers. When a crosslinking agent is optionally used, the agent can also provide additional crosslinks between polymer molecules of the superabsorbent particles and the hydrophilic polymer bound to the cellulose fibers. To take advantage of this favorable attractions between superabsorbent particles and the hydrophilic polysaccharide treated cellulose fibers, the superabsorbent particles used should not be highly crosslinked. The metal crosslink arises as a consequence of an associative interaction (e.g., bonding) between functional groups on the hydrophilic polymers (e.g., carboxy, carboxylate, or hydroxyl groups) and a multi-valent metal species (see description of crosslinking agents above). The superabsorbent particles and the treated cellulose fiber contain hydrophilic polymers that can form metal crosslinks. Suitable multi-valent metal species include metal ions having a valency of three or greater and that are capable of forming an associative interaction with a polymer (e.g., reactive toward associative interaction with the polymer's carboxy, carboxylate, or hydroxyl groups). The polymers are intermolecularly crosslinked when the multi-valent metal species forms an associative interaction with functional groups on two or more polymer molecules. A crosslink may be formed within one polymer molecule or may be formed between two or more polymer molecules.

In the method, the cellulose fibers having superabsorbent particles attached thereto are obtained by mixing the second aqueous mixture including the plurality of superabsorbent particles and treated cellulose with a water-miscible solvent. Suitable water-miscible solvents include water-miscible alcohols and ketones. Representative water-miscible solvents include acetone, methanol, ethanol, isopropanol, and mixtures thereof In one embodiment, the water-miscible solvent is ethanol. In another embodiment, the water-miscible solvent is isopropanol. The volume of water-miscible solvent added to the gel ranges from about 1:1 to about 1:5 water to water-miscible solvent.

In the method, mixing the gel with the water-miscible solvent includes stirring to provide fibers with adhered superabsorbent particles. The mixing step and the use of the water-miscible solvent controls the rate of dehydration and solvent exchange and provides fiber with adhering superabsorbent particles. Mixing can be carried out using a variety of devices including overhead stirrers, Hobart mixers, British disintegrators, and blenders.

### Test Methods

### Free Swell and Centrifuge Retention Capacities

The materials, procedure, and calculations to determine free swell capacity (g/g) and centrifuge retention capacity (CRC) (g/g) were as follows.

Test Materials:

Japanese pre-made empty tea bags (available from Drugstore.com, IN PURSUIT OF TEA polyester tea bags 93 mm×70 mm with fold-over flap. (http:www.mesh.ne.jp/tokiwa/)).

Balance (4 decimal place accuracy, 0.0001 g for air-dried superabsorbent polymer (ADS SAP) and tea bag weights); timer; 1% saline; drip rack with clips (NLM 211); and lab centrifuge (NLM 211, Spin-X spin extractor, model 776S, 3,300 RPM, 120 v).

Test Procedure:

1. Determine solids content of ADS.

2. Pre-weigh tea bags to nearest 0.0001 g and record.

3. Accurately weigh 0.2025 g±0.0025 g of test material (SAP), record and place into pre-weighed tea bag (air-dried (AD) bag weight). (ADS weight+AD bag weight=total dry weight).

4. Fold tea bag edge over closing bag.

5. Fill a container (at least 3 inches deep) with at least 2 inches with 1% saline.

6. Hold tea bag (with test sample) flat and shake to distribute test material evenly through bag.

7. Lay tea bag onto surface of saline and start timer.

8. Soak bags for specified time (e.g., 30 minutes).

9. Remove tea bags carefully, being careful not to spill any contents from bags, hang from a clip on drip rack for 3 minutes.

10. Carefully remove each bag, weigh, and record (drip weight).

11. Place tea bags onto centrifuge walls, being careful not to let them touch and careful to balance evenly around wall.

12. Lock down lid and start timer. Spin for 75 seconds.

13. Unlock lid and remove bags. Weigh each bag and record weight (centrifuge weight).

Calculations:

The tea bag material has an absorbency determined as follows:

Free Swell Capacity, factor=5.78

Centrifuge Capacity, factor=0.50

Z=Oven dry SAP wt (g)/Air dry SAP wt (g)

Free Capacity (g/g):

$$\frac{\begin{matrix}[ ( drip wt ( g ) - dry bag wt ( g ) ) - ( AD SAP wt ( g ) ) ] - \\ ( dry bag wt ( g ) * 5.78 )\end{matrix}}{( AD SAP wt ( g ) * Z )}$$

Centrifuge Retention Capacity (g/g):

$$\frac{\begin{matrix}[ centrifuge wt ( g ) - dry bag wt ( g ) - ( AD SAP wt ( g ) ) ] - \\ ( dry bag wt ( g ) * 0.50 )\end{matrix}}{( AD SAP wt * Z )}$$

### Absorbency Under Load (AUL)

The materials, procedure, and calculations to determine AUL were as follows.

Test Materials:

Mettler Toledo PB 3002 balance and BALANCE-LINK software or other compatible balance and software. Software set-up: record weight from balance every 30 sec (this will be a negative number. Software can place each value into EXCEL spreadsheet.

Kontes 90 mm ULTRA-WARE filter set up with fritted glass (coarse) filter plate. clamped to stand; 2 L glass bottle with outlet tube near bottom of bottle; rubber stopper with glass tube through the stopper that fits the bottle (air inlet); TYGON tubing; stainless steel rod/plexiglass plunger assembly (71 mm diameter); stainless steel weight with hole drill through to place over plunger (plunger and weight=867 g); VWR 9.0 cm filter papers (Qualitative 413 catalog number 28310-048) cut down to 80 mm size; double-stick SCOTCH tape; and 0.9% saline.

Test Procedure:

1. Level filter set-up with small level.

2. Adjust filter height or fluid level in bottle so that fritted glass filter and saline level in bottle are at same height.

3. Make sure that there are no kinks in tubing or air bubbles in tubing or under fritted glass filter plate.

4. Place filter paper into filter and place stainless steel weight onto filter paper.

5. Wait for 5-10 min while filter paper becomes fully wetted and reaches equilibrium with applied weight.

6. Zero balance.

7. While waiting for filter paper to reach equilibrium prepare plunger with double stick tape on bottom,

8. Place plunger (with tape) onto separate scale and zero scale.

9. Place plunger into dry test material so that a monolayer of material is stuck to the bottom by the double stick tape.

10. Weigh the plunger and test material on zeroed scale and record weight of dry test material (dry material weight 0.15 g±0.05 g).

11. Filter paper should be at equilibrium by now, zero scale.

12. Start balance recording software.

13. Remove weight and place plunger and test material into filter assembly.

14. Place weight onto plunger assembly.

15. Wait for test to complete (30 or 60 min)

16. Stop balance recording software.

Calculations:

A=balance reading (g)*−1 (weight of saline absorbed by test material)

B=dry weight of test material (this can be corrected for moisture by multiplying the AD weight by solids %).

AUL (g/g)=A/B (g 1% saline/1 g test material)

The following examples are provided for the purpose of illustrating, not limiting, the invention.

### EXAMPLES

### Example 1

### The Preparation of Representative Guar Gum Treated Cellulose Fibers

In this example, the preparation of representative guar gum treated cellulose fibers is described.

Guar gum (4.0 g) was dissolved in 3200 ml of deionized water. Northern kraft spruce (NKS) pulp (40.0 g) was dispersed in the guar gum solution and oven dried at 105° C. This material was used for binding superabsorbent composite particles.

### Example 2

### The Preparation of a Representative Fibrous Superabsorbent Composite

In this example, the preparation of a representative fibrous superabsorbent composite containing cellulose and commercial superabsorbent particles crosslinked with aluminum sulfate.

Aluminum sulfate octadecahydrate 0.035 g was dissolved in 50 ml of deionized water at 80° C. Guar gum treated cellulose fiber (prepared as described as in Example 1) 1.2 g was then dispersed in the aluminum sulfate solution for 15 minutes. Commercial superabsorbent particles (SANWET IM-4500 from Hoechst Celanese) 2.8 g was added to the fiber slurry and mixed for 2 minutes. To the swollen mass of fiber gel was added 150 ml of isopropanol and mixed for 5 minutes to obtain composite fiber with attached superabsorbent particles. The composite fiber obtained was then filtered. The fiber mass was partially dried in the oven at 66° C. The fiber mass was then fiberized and dried in the oven at 66° C.

T-bag test gave free swell of 39.75 g/g; centrifuge capacity of 19.86 g/g; and AUL of 27.36 g/g (at 0.3 psi) for 0.9% saline solution.

### Example 3

### The Preparation of a Representative Fibrous Superabsorbent Composite

In this example, the preparation of a representative fibrous superabsorbent composite containing cellulose and commercial superabsorbent particles without added crosslinking agent.

Guar gum treated cellulose fiber (prepared as described as in Example 1) 1.2 g was then dispersed in 50 ml of deionized water at 80° C. for 15 minutes. Commercial superabsorbent particles (SANWET IM-4500 from Hoechst Celanese) 2.8 g was added to the fiber slurry and mixed for 2 minutes. To the swollen mass of fiber get was added 150 ml of isopropanol and mixed to obtain composite fiber with attached superabsorbent particles. The composite fiber obtained was the filtered. The fiber mass was partially dried in the oven at 66° C. The fiber mass was then fiberized and dried in the oven at 66° C.

T-bag test gave free swell of 40.50 g/g; centrifuge capacity of 23.54 g/g; and AUL of 28.15 g/g (at 0.3 psi) for 0.9% saline solution.

TABLE 1
Composition and Absorbent Properties of Composite Superabsorbent
Fiber from Synthetic Superabsorbent and Galactomannan Treated Cellulose

| | Superabsorbent particle | Polysaccharide polymer treated cellulose | | Free Swell | CRC | AUL |
|---|---|---|---|---|---|---|
| Sample | (wgt % total wgt) | (wgt % total wgt) | Crosslinking agent/4 g | (g/g) | (g/g) | (g/g) |
| 1 | SANWETIM-4500 (50%) | NKS pulp with 10% GG (50%) | 0.017 g Al₂(SO₄)₃ with wash | 35.3 | 15.62 | 23.31 |
| 2 | SANWETIM-4500 (50%) | NKS pulp with 10% GG (50%) | — | 33.95 | 16.61 | 23.8 |
| 3 | SANWETIM-4500 (70%) | NKS pulp with 10% GG (30%) | 0.017 g Al₂(SO₄)₃ with wash | 39.75 | 19.86 | 27.36 |
| 4 | SANWETIM-4500 (70%) | NKS pulp with 10% GG (30%) | — | 40.5 | 23.54 | 28.15 |

While illustrative embodiments have been illustrated and described, it will be appreciated that various changes can be made therein without departing from the spirit and scope of the invention.

The embodiments of the invention in which an exclusive property or privilege is claimed are defined as follows:

1. Cellulose fibers having superabsorbent particles adhered thereto, comprising cellulose fibers treated with a hydrophilic polysaccharide polymer and having superabsorbent particles adhered thereto, wherein the particles are selected from the group consisting of synthetic polymers, carboxyalkyl cellulose polymers, carboxyalkyl starch polymers, alginates, chitosans, and starches.

2. The fibers of claim 1, wherein the polysaccharide is selected from the group consisting of galactomannan polymers, glucomannan polymers, alginic acids, carageenans, carboxymethyl cellulose, hydroxyethyl cellulose, starch, carboxymethyl starch, and hydroxyethyl starch.

3. The fibers of claim 1, wherein the polysaccharide comprises guar gum.

4. The fibers of claim 1, wherein the particles are synthetic polymers.

5. The fibers of claim 4, wherein the synthetic polymers are selected from the group consisting of polyacrylic acid polymers, polyacrylamide polymers, and polyaspartic acid polymers.

6. Cellulose fibers having superabsorbent particles adhered thereto, comprising cellulose fibers treated with a hydrophilic polysaccharide polymer and a crosslinking agent, wherein the cellulose fibers treated with hydrophilic polysaccharide polymer and crosslinking agent have superabsorbent particles adhered thereto, wherein the particles are selected from the group consisting of synthetic polymers, carboxyalkyl cellulose polymers, carboxyalkyl starch polymers, alginates, chitosans, and starches.

7. The fibers of claim 6, wherein the polysaccharide is selected from the group consisting of galactomannan polymers, glucomannan polymers, alginic acids, carageenans, carboxymethyl cellulose, hydroxyethyl cellulose, starch, carboxymethyl starch, and hydroxyethyl starch.

8. The fibers of claim 6, wherein the polysaccharide comprises guar gum.

9. The fibers of claim 6, wherein the particles are synthetic polymers.

10. The fibers of claim 9, wherein the synthetic polymers are selected from the group consisting of polyacrylic acid polymers, polyacrylamide polymers, and polyaspartic acid polymers.

11. The fibers of claim 6, wherein the crosslinking agent is selected from the group consisting of aluminum (III) compounds, titanium (IV) compounds, bismuth (III) compounds, boron (III) compounds, and zirconium (IV) compounds.

12. The fibers of claim 6, wherein the crosslinking agent is an aluminum (III) compound.
